(19) United States
(12) Patent Application Publication
(10) Pub. No.: US 2012/0177732 A1
(43) Pub. Date: Jul. 12, 2012

(54) CONCENTRATION AND MENTAL PERFORMANCE AMPLIFYING FORMULATION

(76) Inventor: Michael Scott BUCKLEY, Toronto (CA)

(21) Appl. No.: 13/420,409

(22) Filed: Mar. 14, 2012

Related U.S. Application Data
(63) Continuation-in-part of application No. PCT/CA2010/001378, filed on Sep. 3, 2008.

(51) Int. Cl. A61K 9/66; A61P 25/00; A61K 36/76

(52) U.S. Cl. 424/455; 424/769

(57) ABSTRACT

A concentration and mental performance amplifying formulation comprising rhodiola rosea and geranium oil, together with at least one of vinpocetine, phosphatidylcholine, caffeine and Salix alba (White Willow Bark). In another aspect, there is disclosed a concentration and mental performance amplifying formulation comprising: rhodiola rosea, geranium oil, and at least one of anhydrous caffeine and nicotine. In yet another aspect, there is disclosed a concentration and mental performance amplifying formulation as last described which further comprises: Vinpocetine, or Vincamine together with Huperzine-A, or Vinca minor (Periwinkle) together with Huperzine-A; PhosphatidylCholine or Bacopa monnieri (Brahmi) or Mucuna pruriens; Nicotinamide Adenine Dinucleotide Hydrate; and Salix alba (White Willow Bark). Also described is a method of amplifying concentration and mental performance in a human subject by administering such formulations to such a subject.

### RELATED APPLICATIONS

This is a Continuation-In-Part application of International Patent Application No. PCT/CA2010/001378 filed Sep. 3, 2010, which designates the United States of America and claims priority from U.S. Provisional Patent Application No. 61/242,168 filed Sep. 14, 2009, which applications are incorporated entirely herein by reference.

### FIELD OF THE INVENTION

This invention relates to a formulation, which is meant for over the counter sale to consumers who wish to increase their focus and mental performance to make them more productive.

### BACKGROUND OF THE INVENTION

When dopamine, epinephrine, serotonin, and acetylcholine levels are low in humans, an imbalance of neurotransmitters in the brain results with a variety of symptoms including lack of focus, boredom, fatigue, stress, headaches, mood swings, depression, and/or increased aggression. There are many causes of these neurotransmitter level reductions, some of which can be due to genetics, which can result in 1) an inability to produce sufficient neurotransmitters and/or an overly rapid re-uptake of the neurotransmitters after release, which reduces their presence in the synaptic cleft and thus their ability to have the desired effect, 2) reduced nutrient delivery to the brain. Diet can also cause such symptoms due to a lack of sufficient essential precursors in the diet.

Every year, more than 13.7 million Canadians between the ages of 18 and 44 pursue post secondary education or are working to advance their careers in the work force. These people have as their goal “The desire to be great!” For Generation Y, achieving this is possible by attending post secondary institutions or entering the workforce to gain the skills/knowledge for great careers. Generation X is now advancing through the workforce to build great careers. With intense competition at school and work for limited opportunities, many of these people are looking for an edge. These people consume coffee and energy drinks for an energy boost, but the benefits they achieve with these products do not always help them to stay focused, just more alert.

The prior art contains many references to formulations that boost energy, improve cognitive function or blood flow. Typically they include multiple ingredients in various combinations. Examples of patents and published applications that fall in this category include the following: U.S. Pat. No. 6,965,969 which describes a composition and method for treating impaired or deteriorating neurological function; WO 2005/0068890 which describes foods, beverages, condiments, spices and salad dressings with specialized supplements; WO 2005/107779 which describes a nutritional composition that promotes weight loss, burns calories, increases thermogenesis, supports energy metabolism and/or suppresses appetite; US 2006/0211721 which describes a nutraceutical formulation of a cognitive enhancement system; US 2006/0280815 which describes a nutritional composition that promotes weight loss, burns calories, increases thermogenesis, supports energy metabolism and/or suppresses appetite; WO 2007/145993 which describes compositions that enhance brain function; U.S. Pat. No. 6,399,116 describes Rhodiola and uses thereof; and US 2008/0305096 which describes a controlled release formulation that can include among other ingredients, geranium oil, Rhodiola rosea extract and white willow bark extract.

### SUMMARY OF THE INVENTION

The present invention provides a formulation that increases focus and mental performance by increasing neurotransmitter levels, reducing neurotransmitter re-absorption once released, increasing oxygen, glucose, and other nutrient utilization and delivery in the brain in individuals that have difficulty staying focused and commonly suffer from other symptoms (such as: boredom, fatigue, stress, headaches, mood swings and aggression).

The core medicinal ingredients in the formulation are Rhodiola Rosea and Geranium Oil. The Geranium oil also functions as a flavour enhancer. Additional ingredients that contribute to the benefits associated with the formulation are Vinpocetine, Phosphatidylcholine, Anhydrous Caffeine and Salix alba (White Willow Bark).

It is possible to modify the formulations as set out above by including one or more of the following ingredients: Bacopa monnieri (Brahmi) or Mucuna pruriens may replace the Phosphatidylcholine; Vincamine or Vinca minor (Periwinkle) together with Huperzine-A may replace Vinpocetine; Vanillin and/or Peppermint Oil may be added as flaovourants; and Nicotinamide Adenine Dinucleotide Hydrate may be present as an additional ingredient.

The following medicinal ingredients may be combined in amounts that fall within the following ranges:

Rhodiola Rosea: about 0.01 mg to about 600 mg

Geranium Oil: about 0.01 mg to about 600 mg

Vinpocetine or Vincamine or Vinca minor (Periwinkle) together with Huperzine-A: about 0.01 mg to about 100 mg

PhosphatidylCholine or Bacopa monnieri (Brahmi) or Mucuna pruriens: about 0.01 mg to about 600 mg

Anhydrous Caffeine: about 0.01 mg to about 600 mg

Salix alba (White Willow Bark): about 0.01 mg to about 600 mg

Nicotinamide Adenine Dinucleotide Hydrate: about 0.01 mg to about 100 mg.

Vanillin and/or Peppermint Oil: about 0.01 mg to about 100 mg.

Other non-medicinal ingredients that may be included in these formulations include tocopherols concentrate which serve as antioxidants and medium chain triglycerides which serve as emulsifying agents, and glyceryl monosterate may be present as a diluent.

While not wishing to be bound by any particular theory, the present formulation is believed to work in two ways:

1. Increases the bioavailability and effectiveness of neurotransmitters:
a. Increases Dopamine, Epinephrine, Serotonin, and Acetylcholine levels;
b. Inhibits the re-uptake and breakdown of Dopamine and Serotonin once released into the synaptic cleft;
   2. Increases cerebral blood flow:
a. Improves cerebral glucose and oxygen utilization; and
b. Augments delivery of neurotransmitters and other nutrients to the cerebral neurons.

The beneficial results include: improved nutrient delivery and utilization by the brain, and a more balanced level of neurotransmitters. This allows the individual to be more focused, ignore distractions, have reduced stress, and to have an overall increase in mental performance.

### DETAILED DESCRIPTION

The ingredients used in the present formulation are known ingredients with uses that have been documented. The following is a description of each of these ingredients, including alternative names and uses that have been associated with them.

Rhodiola rosea

### Also Known as:

Golden Root, Hongjingtian, King's Crown, Roseroot, Rhodiola, Rosenroot, Rodia riza, Siberian Golden Root, Siberian Rhodiola Rosea, Lignum rhodium.

Scientific Name:

Rhodiola rosea, synonyms Sedum rhodiola, Sedum rosea.

### Family: Crassulaceae.

People Use this for:

Orally, Rhodiola rosea is used for increasing energy, stamina, strength and mental capacity; and as a so-called “adaptogen” to help the body adapt to and resist physical, chemical, and environmental stress. It is also used for improving athletic performance, improving sexual function, depression, anxiety, cardiac disorders such as arrhythmias, and hyperlipidemia. Rhodiola rosea is also used for treating cancer, tuberculosis, and diabetes; preventing cold and flu, swine flu, aging, and liver damage; improving hearing; strengthening the nervous system; enhancing immunity; and shortening recovery time after prolonged workouts.

Mechanism of Action:

The applicable part of Rhodiola rosea is the root. Rhodiola rosea contains over 30 compounds including phenlyethanoids, phenylpropanoids, flavonoids, cyanoglycosides, monoterpenes, and triterpenes (15718).

The phenylpropanoid glycoside called salidroside is thought to be responsible for many of the stimulant or “adaptogenic” effects of Rhodiola rosea (6877, 13028). It is also sometimes referred to as rhodioloside or rhodosine (13028). Other constituents isolated from Rhodiola rosea include rhodioniside, rhodioloside A-E, rhodiolin, rosin, rosavin, rosarin, rosiridin, rosiridol, rhodalgin, acetylrhodalgin, and lotaustralin (13028, 13059, 16410). It is thought that these constituents might also be involved in Rhodiola rosea's adaptogenic effects (13028).

Some Rhodiola rosea products are standardized based on rosavin content and salidroside content. Rosavin is specific to Rhodiola rosea and distinguishes it from other species in the Rhodiola genus (13028).

Rhodiola rosea also contains the tannins gallic acid and caffeic acid, as well as chlorogenic acid and flavonoids such as catechins and proanthocyanidins (13028, 13059, 15713). These compounds are likely responsible for the antioxidant activity of Rhodiola rosea extracts (13028). In vitro, salidroside decreases apoptosis of neuroblastoma cells exposed to hydrogen peroxide, suggesting that it might protect against oxidative stress (15714).

The amounts of active constituents in Rhodiola rosea can vary significantly depending on the source of plant material and plant material collection period (15713). Animal studies are reported to show protection from stressors such as cold and radiation, increased work capacity, decreased fatigue and improved learning and memory (6877). Rhodiola rosea extracts demonstrate antiarrhythmic properties and protection against reperfusion injury after ischemia.

Rhodiola rosea appears to have significant central nervous system activity. In animal models, a Rhodiola rosea extract containing 3% rosavin and 1% salidroside has antidepressant, anxiolytic, and stimulant effects (15716). Rhodiola rosea extracts also demonstrate potential for improving learning and memory (3198, 6877).

Geranium Oil

### Also Known as:

### Aetheroleum Pelargonii, Algerian Geranium Oil, Bourbon Geranium Oil, Geranium, Moroccan Geranium Oil, Oleum Geranii, Rose Geranium Oil, Pelargonium Oil.

Scientific Name:

Pelargonium graveolens.

### Family: Geraniaceae.

People Use this for:

Orally, geranium oil is used for neuropathic pain and diarrhea.

Mechanism of Action:

The applicable part of geranium is the oil that is distilled from the stem and leaf. Geranium oil is used for relieving neuropathic pain; however, the mechanism is not known. (4912)

Vinpocetine

### Also Known as:

AY-27255, Cavinton, Ethyl Apovincaminate, Ethylapovincaminoate, Vinca minor, Periwinkle, RGH-4405, TCV-3b, Vinpocetin.

Scientific Name:

Eburnamenine-14-carboxylic acid; Ethyl Ester.

People Use this for:

Orally, vinpocetine is used for enhancing memory, improving cerebral blood flow, improving cerebral oxygen and glucose utilization, protecting against age-related cognitive decline and Alzheimer's disease, treating cerebrovascular disease, preventing post-stroke morbidity and mortality, treating organic psychosyndromes, treating intractable tumoral calcinosis in people undergoing hemodialysis, decreasing stroke risk, treating menopausal symptoms, chronic fatigue syndrome (CFS), seizure disorders, and preventing motion sickness. Intravenously, vinpocetine is injected for treating seizure disorders and stroke.

Mechanism of Action:

Vinpocetine is a synthetic derivative of apovincamine, a compound found in the periwinkle plant, Vinca minor. Some studies indicate that vinpocetine might enhance cerebral blood flow without affecting peripheral blood flow (1786, 1793).

Preliminary evidence indicates that vinpocetine stimulates cerebral metabolism and increases glucose and oxygen consumption by the brain (10827). Potential mechanisms for the nootropic-like effects of vinpocetine include indirect or direct cholinergic activity, augmented norepinephrine effects on cortical cyclic adenosine monophosphate (AMP), and increased turnover of brain catecholamines (1800). It might also improve microcirculation in the brain and increase cerebral blood flow by improving red blood cell deformability, reducing cerebral vascular resistance, and inhibiting platelet aggregation (10827). Vinpocetine inhibits drug-induced platelet aggregation (1801). Pharmacological effects that might be useful in treating stroke include a possible neuroprotective and anticonvulsant effect by blocking voltage-gated sodium channels. It also might protect neurons by enhancing the effect of adenosine in preventing hypoxia. Animal studies suggest that vinpocetine decreases neuronal death in ischemia and decreases the size of cerebral infarction in experimental strokes (10728). The bioavailability of vinpocetine varies from 7-57%; food significantly enhances absorption (1802).

Phosphatidylcholine

### Also Known as:

1,2-diacyl-:ussn:ueglycero-3-phosphocholine, Lipodissolve, Lipolight, Lipolyse, Lipotherapy, Phospholipid, Phospholipon, Polyenylphosphatidylcholine, Lecithin, Soy, PtdCho. Phosphatidylethanolamine

Phosphatidylcholine is present to provide a source of acetylcholine. There are many alternatives that will provide this and the following are some examples of substances that may be used: Acetyl-L-Carnitine, Alpha-GPC, Betaine, Choline, Phosphatidylserine, Citicoline, Choline Bitartrate, Choline Chloride, Choline Citrate, Intrachol, L-Choline, Lipotropic Factor, Methylated Phosphatidylethanolamine.

Scientific Name:

### None.

People Use this for:

Orally, phosphatidylcholine is used for treating anxiety, eczema, gallbladder disease, hepatitis, manic-depressive illness, peripheral vascular disorders, hyperlipidemia, improving ultrafiltration in peritoneal dialysis, tardive dyskinesia, premenstrual syndrome, memory loss, Alzheimer's disease, immunodepression, and preventing aging. Intravenously, phosphatidylcholine is used for angina, lipid atheromas, fat embolism, hypercholesterolemia, liver disease, and fatty plaque deposits. Subcutaneously, phosphatidylcholine is used for lipoma, periorbital fat pad herniation, xanthelasmas, and removing fatty deposits for cosmetic purposes.

Mechanism of Action:

When taken orally, phosphatidylcholine is absorbed rapidly and reaches maximum serum concentrations in 8-12 hours (15626). Phosphatidylcholine is the largest reservoir of choline in the body (15626). Choline is a precursor to acetylcholine (5228).

Caffeine

### Also Known as:

### Anhydrous Caffeine, Caffeine and Sodium Benzoate, Caffeine Anhydrous, Caffeine Citrate, Citrated Caffeine, Methylxanthine, Trimethylxanthine.

Anhydrous Caffeine is present to provide a source of caffeine. There are many alternatives that will provide this and the following are some examples: Guarana, Green Tea, Cocoa, Coffee, Black Tea, Cola Nut, Mate, Oolong Tea, Pu-Erh Tea, Sanicle, Theanine, Wahoo.

Scientific Name:

1,3,7-trimethylxanthine.

People Use this for:

Orally, caffeine is used in combination with analgesics and ergotamine for treating migraine headaches. It is used orally with analgesics for simple headaches and preventing and treating postoperative and postdural puncture headaches. It is also used orally for asthma, gallbladder disease, attention-deficit hyperactivity disorder (ADHD), neonatal apnea, hypotension, increasing mental alertness, and enhancing athletic performance. Caffeine is used for weight loss and type 2 diabetes. Very high doses are used as euphoriants, often in combination with ephedrine as an alternative to illicit stimulants. Topically, caffeine cream preparations have been used for reducing erythema and itching in dermatitis.

Rectally, caffeine is used in combination with ergotamine for migraine headaches.

Parenterally, caffeine is used for postoperative and postdural puncture headache, neonatal apnea, acute respiratory depression, and as a diuretic. It is also used for extending the length of seizure with electroconvulsive therapy. In foods, caffeine is used as an ingredient in soft drinks, energy drinks, and other beverages.

Mechanism of Action:

Caffeine is a methylxanthine compound and is structurally related to theophylline, theobromine, and uric acid (6372). It is 100% bioavailable after oral administration and is metabolized principally in the liver to paraxanthine, theophylline, and theobromine (6370). The half-life of caffeine is about six hours (8644).

Caffeine stimulates the central nervous system (CNS), heart, muscles, and possibly the pressor centers that control blood pressure (2722). Possible mechanisms include adenosine receptor blockade and phosphodiesterase inhibition (2722). By blocking adenosine receptors, caffeine is thought to increase the release of neurotransmitters such as dopamine (6370). Caffeine also decreases airway resistance and stimulates respiration, via adenosine receptor blockade and phosphodiesterase inhibition (11836). It has also been proposed that caffeine may decrease GABA and serotonin signaling (6370).

Caffeine can have positive inotropic and chronotropic effects on the heart (11836). Caffeine can also acutely elevate both diastolic and systolic blood pressure, but might not have this effect in habitual users (2722).

Caffeine's CNS stimulant effects are thought to improve vigilance and psychomotor performance (2720,10205). For improving athletic performance, caffeine has been shown to decrease perceived levels of exertion, which enables the athlete to feel less tired and increase their performance (6370). Caffeine seems to enhance muscle metabolism and increases time to exhaustion and oxygen deficit, which may lead to better performance (8646).

Caffeine has been reported to cause increases and decreases in blood glucose (12374). For preventing Parkinson's disease, caffeine may protect dopaminergic neurons in the brain. This effect appears to be related to modulation of adenosine receptors (10201). This may result in a reduction in the clinical expression of Parkinsonism (6022).

Evidence suggests that tolerance to caffeine's neuroendocrine and cardiovascular effects may develop during consumption throughout the day, but tolerance appears to be lost during overnight abstinence of caffeine (6372). Preliminary evidence also suggests caffeine may increase plasma levels of cortisol and adrenocorticotrophic hormone (ACTH), decrease levels of extracellular potassium, and increase levels of intracellular calcium in skeletal muscle; but the mechanisms are poorly understood (6370).

Caffeine increases resting energy expenditure (REE) and cellular thermogenesis. It also causes an increase in nonoxidative fatty acid turnover and lipid oxidation; however, the net effect on lipid oxidation is small. The effects of caffeine on energy expenditure and lipid metabolism seem to be mediated by both sympathetic and nonsympathetic mechanisms (13733).

White Willow Bark (Salix alba)

### Also Known as:

Basket Willow, Bay Willow, Black Willow, Black Willowbark, Black Willow Extract, Brittle Willow, Crack Willow, Daphne Willow, European Willow, European Willow Bark, Knackweide, Laurel Willow, Lorbeerweide, Organic Willow, Osier Rouge, Purple Willow, Pupurweide, Purple Osier, Purple Osier Willow, Pussy Willow, Reifweide, Salicis Cortex, Silberweide, Violet Willow, Weidenrinde, White Willow, White Willow Bark, White Willow Extract, Willow Bark Extract.

White Willow Bark is present to provide a source of salicylates. There are many alternatives that will provide this and the following are some examples: Aloe, Ashwaghanda, Aspen, Black Haw, Cranberry, German Sarsasparilla, Isatis, Meadow Sweet, Poplar, Senega, Tamarind, Wintergreen, Yarrow.

Scientific Name:

Salix alba; Salix daphnoides; Salix fragilis; Salix nigra; Salix pentandra; Salix purpurea; other Salix species.

### Family: Salicaceae.

Known Uses:

Orally, willow bark is used for headache, pain, myalgia, osteoarthritis, dysmenorrhea, gouty arthritis, ankylosing spondylitis, rheumatoid arthritis (RA), and gout. It is also used for fever, common cold, influenza, swine flu, and weight loss.

Mechanism of Action:

Willow bark is the bark of salix tree species such as the white willow. Willow bark constituents include flavonoids, tannins, and salicylates. The active constituent of willow bark is thought to be salicin. Salicin is metabolized to salicyl alcohol and then to salicylic acid. From there, metabolism is the same as aspirin (12808).

An ethanolic extract of willow bark seems to inhibit cyclooxygenase (COX)-2 mediated prostaglandin release, but it doesn't seem to directly affect COX-1 or COX-2 activity. Constituents of willow bark other than salicin may have lipoxygenase-inhibiting and antioxidant effects that could contribute to its analgesic effect (6456, 12476).

Preliminary research suggests that willow bark extracts have analgesic, anti-inflammatory, and antipyretic effects (12476). Willow bark inhibits platelet aggregation, but to a lesser degree than aspirin (12810).

Huperzine A

Also known as HupA, Huperzina A, Huperzine, Huperzine-A, Selagine, Sélagine.

Scientific Name:

### Huperzine A

People Use this for

Orally, huperzine A is used for Alzheimer's disease, memory and learning enhancement, age-related memory impairment, increasing alertness and energy, protection from neurotoxic agents including organophosphate nerve gases, glutamate toxicity, and for treating myasthenia gravis.

Mechanism of Action:

Huperzine A is an alkaloid isolated from Chinese club moss, Huperzia serrata and from Lycopodium selago. It is an optically active stereoisomer. Only the levorotatory-isomer is pharmacologically active (3561). Huperzine A is thought to be beneficial in dementia, memory impairment, and myasthenia gravis due to its effects on acetylcholine levels (3133, 3134, 3135, 3136). It is a reversible inhibitor of acetylcholinesterase (AChE) for up to three hours, and crosses the blood-brain barrier. It produces a variable degree of acetylcholine elevation in different areas of the brain, with maximal values in the frontal and parietal cortex (125% and 105% respectively), and 22-65% in other brain regions (3141). It might be more specific for AChE and have a longer duration of action than AChE inhibitors such as tacrine (Cognex) or donepezil (Aricept), which are marketed as prescription drugs for Alzheimer's disease (3131, 3132). In animal studies, huperzine A was found to be 64 times more potent than tacrine. It also is more bioavailable and penetrates the blood-brain barrier better than tacrine (3561). Huperzine A protects neurons against toxic levels of glutamate by blocking glutamate-induce neuronal calcium influx and cell death (3131, 3561). Although it has low affinity, huperzine A is also a cerebral cortex N-methyl-D-aspartate (NMDA) receptor antagonist (3129, 3137). It might also protect against seizures and neuropathological changes caused by exposure to organophosphate nerve agents such as soman, by protecting peripheral and central stores of acetylcholine (3137). Natural Medicines Comprehensive Database

Vanilla (Vanillin)

### Also Known As:

### Bourbon Vanilla, Common Vanilla, Madagascar Vanilla, Mexican Vanilla, Reunion Vanilla, Tahiti Vanilla, Tahitian Vanilla, Vainilla, Vanillin.

Scientific Name:

Vanilla planifolia, synonyms Vanilla fragrans, Myrobroma fragrans; Vanilla tahitensis.

### Family: Orchidaceae.

People Use this for:

Orally, vanilla is used as an aphrodisiac, antiflatulent, antipyretic, and stimulant. In foods and beverages, vanilla is used as a flavoring agent. It is added to foods to reduce the amount of sugar needed for sweetening and inhibit the development of dental caries. In manufacturing, vanilla is used as a flavoring agent in syrups for pharmaceutical use. It is also used as a fragrance in perfumes.

Mechanism of Action:

The applicable part of vanilla is the fruit. Although the constituent vanillin is primarily responsible for the flavor of vanilla (11), over 150 aromatic compounds contribute to its fragrance (11, 6). The catechin content of vanilla shows evidence of an anti-caries effect (6). In controlled studies, meals flavored with vanilla provided a higher degree of satisfaction than identical meals without vanilla flavoring (6).

Contact dermatitis associated with the vanilla plant is thought to be due to the calcium oxalate crystals in the plant (6).

Cowhage (Mucuna Pruriens)

### Also Known As:

Atmagupta, Cowitch, Cow-Itch Plant, Dolichos Pruriens, Feijao Macaco, HP 200, HP-200, Kapi Kacchu, Kapikachchhu, Kapikachhu, Kapikachu, Kaunch, Kawach, Kawanch, Kevanch, Kiwach, Mucuna, Mucuna Prurient, Mucuna Prurita, Ojo de Buey, Ojo de Venado, Pica-Pica, Velvet Bean.

Scientific Name:

Mucuna pruriens; Mucuna pruriens var. hirsuta, synonyms Mucuna hirsuta, Stizolobium hirsutum.

### Family: Fabaceae/Leguminosae.

People Use this for:

Orally, cowhage is used for Parkinson's disease, anxiety, arthritis, hyperprolactinemia, and for parasitic infections. It's also used as an analgesic for pain, for fever, to induce vomiting, and as an aphrodisiac. Cowhage is also used prophylactically as snakebite remedy.

Topically, cowhage is used as a rubefacient or counterirritant for rheumatic conditions, myalgias, to stimulate cutaneous blood flow in paralytic conditions, and to treat scorpion stings.

Mechanism of Action:

The applicable parts of cowhage are the bean or seed and the hair on the bean pod.

Cowhage is thought to work for Parkinson's disease because it contains a significant amount of levodopa (L-dopa). The whole cowhage bean contains about 3% to 6% L-dopa (7020, 7021). The inner layer (endocarp) of the pericarp, which has also been studied in patients with Parkinson's disease, usually contains the highest amount of L-dopa, about 5.3% (7020). Symptoms of Parkinson's disease occur in patients due to a depletion of the neurotransmitter dopamine. L-dopa is a precursor to dopamine. To be effective for Parkinson's disease, L-dopa must cross the blood-brain barrier where it is then decarboxylated to dopamine. However, the majority of L-dopa is metabolized peripherally and probably less than 1% actually reaches the brain (15).

Some powdered cowhage seed preparations containing L-dopa seem to lessen symptoms of Parkinson's disease at a relatively low dose, compared to conventional L-dopa products. So there is some speculation that constituents other than levodopa Natural Medicines Comprehensive Database darkening of bodily fluids, muscle cramps, headache, and priapism (15). However, these effects have not yet been reported for cowhage. Ingestion of hairs from the bean pod or seed can result in significant mucosal irritation and should be avoided.

Topically, hairs from the cowhage bean pod or seed can cause severe itching, burning, inflammation, and erythematous macular rashes (18, 6898). Symptoms resolve spontaneously within several hours, but may also be relieved with antihistamines (6898). The hairs can be removed from the skin by washing, but the hairs can also be retained, and transferred to other people, in fabrics and carpets. Clothing and other materials that come in contact with the cowhage hairs should also be thoroughly washed (6898).

Peppermint

### Also Known as:

Black Peppermint, Bo He, Brandy Mint, Chinese Peppermint, Corn Mint, Extract of Mentha Piperita, Extract of Peppermint, Extract of Peppermint Leaves, Extrait de Feuilles de Menthe de Poivrée, Extrait de Mentha Piperita, Extrait de Menthe Poivrée, Feuille de Menthe Poivrée, Field Mint, Herba Menthae, Huile de Mentha Piperita, Huile de Menthe Poivrée, Huile Essentielle de Menthe Poivrée, Lamb Mint, Menta Piperita, Mentha Oil, Mentha Piperita Extract, Mentha Piperita Oil, Menthae Piperitae Aetheroleum, Menthae Piperitae Folium, Menthe, Menthe Anglaise, Menthe Poivrée, Menthe Pouliot, Menthe Sauvage, Menthe Verte, Menthol, Mint, Mint Balm, Oil of Peppermint, Paparaminta, Peppermint Essential Oil, Peppermint Extract, Peppermint Leaf, Peppermint Leaf Extract, Peppermint Oil, Sentebon, Western Peppermint, Yerba Buena. CAUTION: See separate listings for Aromatherapy, Diviner's Sage, English Horsemint, Japanese Mint, Perilla, Spearmint, and Wild Mint.

Scientific Name:

Mentha×piperita, synonym Mentha lavanduliodora; Mentha arvensis; Mentha halpocalyx.

### Family: Lamiaceae/Labiatae.

People Use this for:

Orally, peppermint is used for the common cold, cough, inflammation of the mouth and pharynx, sinusitis, fever, liver and gallbladder complaints, irritable bowel syndrome (IBS), cramps of the upper gastrointestinal (GI) tract and bile ducts, dyspepsia, fever, flatulence, and for tension headache. It is also used for nausea, Natural Medicines Comprehensive Database vomiting, morning sickness, respiratory infections, dysmenorrhea, diarrhea, small intestinal bacterial overgrowth, and as a stimulant. Topically, peppermint oil is used for headache, myalgias, neuralgias, toothache, oral mucosa inflammation, rheumatic conditions, pruritus, urticaria, bacterial and viral infections, as an antispasmodic in barium enemas, and for repelling mosquitoes. As an inhalant, peppermint oil is used as an aromatic, for symptomatic treatment of cough and colds, and as an analgesic for pain. In foods and beverages, peppermint is a common flavoring agent. In manufacturing, peppermint oil is used as a fragrance component in soaps and cosmetics, and as a flavoring agent in pharmaceuticals. vomiting, morning sickness, respiratory infections, dysmenorrhea, diarrhea, small intestinal bacterial overgrowth, and as a stimulant. Topically, peppermint oil is used for headache, myalgias, neuralgias, toothache, oral mucosa inflammation, rheumatic conditions, pruritus, urticaria, bacterial and viral infections, as an antispasmodic in barium enemas, and for repelling mosquitoes. As an inhalant, peppermint oil is used as an aromatic, for symptomatic treatment of cough and colds, and as an analgesic for pain. In foods and beverages, peppermint is a common flavoring agent. In manufacturing, peppermint oil is used as a fragrance component in soaps and cosmetics, and as a flavoring agent in pharmaceuticals.

Mechanism of Action:

The applicable parts of peppermint are the aerial parts and oil. Peppermint oil is obtained by distilling the aerial parts of peppermint. Peppermint oil is a complex mixture of compounds, including 35% to 70% menthol, 15% to 30%% menthone, and 4% to 14% menthyl acetate (13413). However, pharmaceutical grade peppermint oil is typically standardized to contain at least 44% menthol. Peppermint oil contains 1% to 4% pugelone, a neuro- and hepatotoxin. However, there are methods to reduce the pugelone content. Concentrations of pugelone below 1% are considered safe (13413). Peppermint leaf and oil contain acetaldehyde, amyl alcohol, menthyl esters, limone, pinene, phellandrene, cadinene, and dimethyl sulfide. Trace constituents include alpha-pinene, sabinene, terpinolene, ocimene, gamma-terpinene, fenchene, alpha and beta-thujone, citronellol, and other compounds (13413). Peppermint oil is used for irritable bowel syndrome (IBS) due to its antispasmodic effects. It seems to reduce slow wave frequency in the small intestine, which slows peristaltic movement (13398). The antispasmodic activity appears to result from direct relaxing effects on the gastrointestinal (GI) tract smooth muscle, characteristic of calcium antagonist action. Peppermint oil may also inhibit potassium depolarization induced responses in the intestine (6744, 11775). This is thought to prevent the hypercontractility that is commonly found in patients with IBS. Peppermint oil may help relieve esophageal spasms by reducing esophageal contractions and improving the uniformity of contractions. Peppermint oil does not affect lower esophageal sphincter pressure (13415).

Preliminary evidence suggests that peppermint oil in combination with caraway oil can reduce gastroduodenal motility when administered orally in enteric-coated capsules (6742). Menthol and other peppermint oil constituents are rapidly absorbed in the proximal intestine resulting in upper gastrointestinal effects such as relaxation of the lower esophageal sphincter. For peppermint oil to exert effects on the lower intestine, it must pass through the upper gastrointestinal tract unmetabolized. Enteric coated peppermint oil formulations prevent upper gastrointestinal tract metabolism (11780). Preliminary research suggests that administering menthol-beta-D-glucuronide orally as a prodrug might deliver menthol to the large intestine. In the intestine, menthol-beta-D-glucuronide is hydrolyzed by bacterial beta-D-glucuronidases to menthol, possibly increasing its usefulness in treating diseases such as ulcerative colitis and Crohn's disease (11776). For pain in myalgias and neuralgias, menthol in topical peppermint oil is thought to have a direct inhibitory effect on the sensitized pain receptors. It might also act centrally to alter pain perception (11781). Peppermint oil has antiviral and antibacterial activities in vitro (13413, 13414). The menthol constituent of peppermint is active against fungal microorganisms that cause onychomycosis such as Trichophyton rubrum, Trichophyton mentagrophytes, Microsporum canis, Epidermophyton floccosum, and Epidermophyton stockdale (13447). Preliminary research suggests that luteolin-7-O-rutinoside from peppermint leaf can inhibit histamine release (12730). Laboratory models of allergic rhinitis suggest that peppermint leaf extract might relieve nasal symptoms (12733). Preliminary research suggests that peppermint leaf might be hepatotoxic in high doses (12731). Other preliminary research suggests that peppermint leaf tea might lower testosterone levels and decrease spermatogenesis in male animals (12732).

Brahmi

### Also Known as:

Andri, Bacopa, Herb of Grace, Herpestis Herb, Indian Pennywort, Jalanimba, Jal-Brahmi, Jalnaveri, Nira-Brahmi, Sambrani Chettu, Thyme-Leave Gratiola. CAUTION: See separate listing for Gotu Kola.

Scientific Name:

Bacopa monnieri, synonym Bacopa monniera; Herpestis monniera; Moniera cuneifolia.

### Family: Scrophulariaceae.

People Use this for:

Orally, brahmi is used to aid learning, and for anxiety, memory problems, attention deficit-hyperactivity disorder (ADHD), allergic conditions, and irritable bowel syndrome. Brahmi has also been used orally for treating backache, hoarseness, mental illness, epilepsy, rheumatism, sexual dysfunction in both men and women, as a nerve tonic, cardiotonic, and as a diuretic.

Mechanism of Action:

The applicable part of brahmi is the leaf. Pharmacological activity of brahmi is attributed to the saponin bacoside and bacopasaponin constituents (10060, 10061). Some evidence suggests purified bacosides A and B may facilitate learning ability and cognitive performance. Possible mechanisms for cognitive improvement include modulation of acetylcholine release, choline acetylase activity, and muscarinic cholinergic receptor binding (10058). Brahmi may also act as a mast cell stabilizer for allergic conditions (10060).

NADH

### Also Known as:

### B-DPNH, BNADH, Coenzyme 1, Enada, NAD, Reduced DPN, Reduced Nicotinamide Adenine Dinucleotide.

Scientific Name:

### Nicotinamide Adenine Dinucleotide Hydrate.

People Use this for:

Orally, NADH is used for improving mental clarity, alertness, and concentration; improving memory; cellular energy; for antioxidant effects; chronic fatigue syndrome (CFS); depression; jet lag; hypertension; Alzheimer's disease; Parkinson's disease; improving athletic endurance; enhancing energy; improving DNA repair; enhancing immune function; reducing aging; protecting the liver from alcohol damage; preventing alcohol-induced inhibition of testosterone; lowering cholesterol levels; and protecting against zidovudine (AZT) toxicity. Intravenously, NADH is used as an IM or IV injection for Parkinson's disease and depression.

Mechanism of Action:

NADH is the reduced form of NAD (nicotinamide adenine dinucleotide), a coenzyme necessary to dehydrogenate primary and secondary alcohols (3082). In dehydrogenation, NAD acts as a hydrogen acceptor, forming NADH. NADH, in turn, serves as a hydrogen donor in the respiratory chain. NADH is an essential intermediate in the cellular processes that generate energy from glucose in the form of ATP. Some evidence suggests oral NADH reduces blood pressure, total cholesterol, and low-density lipoprotein (LDL) (3083). Preliminary evidence suggests that NADH might help people with chronic fatigue syndrome (CFS) by triggering energy production through ATP generation (8267). Preliminary research suggests that NADH might protect against age-related hypertension, possibly by lowering lipid peroxidation and free radical formation (8260). NADH has been proposed as a therapeutic agent for people with Parkinson's disease because evidence suggests it might increase tyrosine hydroxylase activity and dopamine production (3085, 3086, 3088, 3091).

Periwinkle

### Also Known as:

Common Periwinkle, Earlyflowering, Evergreen, Lesser Periwinkle, Myrtle, Petite Pervenche, Small Periwinkle, Vincae minoris herba, Wintergreen. CAUTION: See separate listings for Madagascar Periwinkle and Wintergreen.

Scientific Name:

Vinca minor.

### Family: Apocynaceae.

People Use this for:

Orally, periwinkle is used for “brain health” (increasing cerebral circulation, supporting brain metabolism, increasing mental productivity, preventing memory and concentration impairment and feebleness, improving memory and thinking capacity, preventing premature aging of brain cells, and geriatric support). It also is used orally for mucous membrane inflammation, diarrhea, vaginal discharge, “blood-purification,” throat ailments, tonsillitis, angina, sore throat, intestinal inflammation, toothache, edema, promoting wound healing, improving immune function, as a diuretic, sedative, antihypertensive, and hemostatic remedy.

Mechanism of Action:

The applicable parts of periwinkle are the above ground parts. Periwinkle contains pharmacologically active, toxic alkaloids including vincristine which have cytotoxic and neurological actions and can injure liver and kidneys (513). Periwinkle may have astringent activity (19). The constituent, vincamine, has hypotensive activity (19). In animals, periwinkle causes leukocytopenia, lymphocytopenia, and lowers alpha-1, alpha-2, and gamma-globulin levels presumably due to immune suppression (2). The periwinkle constituent, vincamine, can be converted in the laboratory to the compound vinpocetine which is marketed as a dietary supplement (1799).

Nicotine

Nicotine is an alkaloid found in the nightshade family of plants (Solanaceae) that constitutes approximately 0.6-3.0% of the dry weight of tobacco,[1][2] with biosynthesis taking place in the roots and accumulation occurring in the leaves.

In low concentrations the substance acts as a stimulant in mammals.

Pharmacokinetics

As nicotine enters the body, it is distributed quickly through the bloodstream and crosses the blood-brain barrier reaching the brain. The elimination half-life of nicotine in the body is around two hours.[14]

Pharmacodynamics

Nicotine acts on the nicotinic acetylcholine receptors, specifically the ganglion type nicotinic receptor and one CNS nicotinic receptor. The former is present in the adrenal medulla and elsewhere, while the latter is present in the central nervous system (CNS). In small concentrations, nicotine increases the activity of these receptors. Nicotine also has effects on a variety of other neurotransmitters through less direct mechanisms.

The following examples are offered by way of illustration of the present invention, and not by way of limitation.

### Example 1

A formulation in accordance with the invention was prepared as follows comprising the following ingredients:

Licithin—140 mg

Rhodiola Rosea (3% Rosavins, 1% Salidrosides)—powder 100 mg

Geranium Oil—liquid 100 mg

Vinpocetine—powder 3 mg

Phosphatidylcholine (Epicuron Lecithin 35% PPC)—liquid 140 mg

Anhydrous Caffeine—powder 100 mg

Salix alba (White Willow Bark)—powder 50 mg

All ingredients were purchased in bulk form and measured out using standard lab procedures and weighed using a Sartorius™ GE812 balance. The desired amount of each ingredient was then placed into an empty size 00 capsule (capsule volume capacity of 0.95 ml). Once all ingredients were placed into the capsule, the capsule was sealed.

The dosage recommendation for an adult for the concentration and mental performance amplifying formulation is one (1) to two (2) capsules as needed every 6-10 hours. No more than 4 capsules per day are recommended.

The above capsules were taken as follows:
A male took two capsules in the morning with a breakfast comprising a protein shake consisting of whey protein, milk, yogurt, oatmeal and strawberries.

Approximately 10-20 minutes after taking the two capsules, the person experienced the following:
An increase in energy (slightly elevated heart rate, though not to the same extent as taking caffeine of an equivalent dosage on its own)

After approximately 30 minutes, the person reported the following experiences:
Heart rate tended to return to normal levels;
An increased desire to complete work and chores that he had been putting aside; if he had been putting off working, he found himself sitting at his computer completing the work he was avoiding;
If he had been putting off chores, he was now doing them;
He was able to ignore distractions that were taking place around him, and focus on the tasks at hand;
Increased productivity in accomplishing tasks; no longer sitting procrastinating away the day;
Thinking more clearly;
Relaxed, increased patience, tolerance, less frustration;
No experience of hyperactivity or jittery symptoms normally associated with the consumption of commercial energy drinks or coffee;
Increased alertness and significant energy increase;
The feeling lasted for 6 to 10 hours and at no point did he feel an energy crash that comes with sugar or energy drinks;
Increased focus on work tasks such that he found he had not realized that an hour or two had gone by as he had been so focused on work and accomplishing a great deal; and
Increased desire to complete tasks and to engage in exercise.

The person also reported the following negative experiences after taking the formulation for a number of days:
Sometimes he forgot to eat, which sometimes led to an energy drain later in the day, due to low blood sugar levels, however if he ate properly throughout the day he did not experience this reaction.

### Comparative Example 2

In this example, a variation of the formulation tested in Example 1 was tested.

The formulation tested was as follows:

| Raw materials | Dosage (mg) |
|---|---|
| Rhodiola Rosea 3% Rosavins 1% Salidrosides | 190.0 |
| Caffeine Anhydrous | 80.0 |
| Geranium Oil | 62.5 |
| Phosphatidylcholine | 100.0 |
| Medium Chain Triglycerides | 102.5 |
| Mixed Tocopherols | 5.0 |
| Glycerol Monostearate | 20.0 |
| Total | 560.0 |

This formulation does not contain vinpocetine or Salix alba. It does contain excipients that are typically found in formulations of this type. The last three ingredients are excipients that are commonly used in this type of formulation. Others may be used and the choice would be readily apparent to the person skilled in the art of formulation.

As for the experience of taking this formulation, it was not equivalent to the experience found with the formulation in Example 1.

1-2 capsules were taken at various times throughout the day (as was felt needed); sample group consisted of 10 normal healthy individuals (males and females between the ages of 23-39);
After about 30 minutes did experience an increased desire to do work, complete tasks;
Able to ignore distractions that are taking place around them, and focus on the tasks at hand;
Increased productivity in accomplishing tasks;
Thinking more clearly;
Relaxed, increased patience, tolerance, less frustration;
No experience of hyperactivity or jittery symptoms normally associated with the consumption of commercial energy drinks or coffee;
Experience lasted for 4-6 hours;
Many were unsure if the experiences they had were associated with taking the product;
If an individual had been experiencing fatigue, he/she did not experience a significant increase in energy, so the fatigue feelings were not significantly reduced;
Some individuals experienced minor headaches;
Some individuals experienced fatigue later in the day due to failure to eat properly throughout the day (when eating properly, this was not experienced).

The formulation of Example 1 was reported to have had a more beneficial effect and also seemed to have been a more pleasant experience because of the variation in the ingredients.

On a volume basis it is anticipated that the active ingredients in the concentration and mental performance amplifying formulation of this invention can be formulated using a process patented by Pfizer Inc that involves liquification of the ingredients to produce liquid containing capsules that can be absorbed more rapidly than other oral forms used for dosage administration, such as tablets and gel capsules. It should be noted that the formulation could be administered in many different forms and the preparation of such different forms is well within the common general knowledge of those skilled in the art. While the present description does not reference foods, or beverages or supplements of any sort, such preparations could be used to deliver the formulation of this invention.

A typical recommended dosage for an adult male or female would be 1 to 2 liquid capsules with food, which would be effective for about 8 hours. The dosage can be taken two times daily as needed up to a maximum recommended dosage of 4 to 6 capsules per day.

The following tables provide additional examples of specific formulations that include alternatives to the previous formulations.

| | | | RM* supplied |
|---|---|---|---|
| | Concen- | Dosage | by (Capsugel |
| Actives | tration | (mg) | or Customer) |
| Rhodiola Rosea 3% Rosavins | 3.00% | 100.000 | Capsugel |
| Caffeine Anhydrous 99% | 99.00% | 100.000 | Capsugel |
| Geranium Oil | 25.00% | 100.000 | Capsugel |
| Mucuna Pruriens | | 100.000 | Capsugel |
| White Willow Bark Extract 15% | 15.00% | 50.000 | Capsugel |
| Salicin | | | |
| Vincamine | | 10.000 | Capsugel |
| Vanillin | | 10.000 | |
| Nicotinamide Adenine Dinucleotide | | 5.000 | |
| Hydrate | | | |
| Peppermint Oil 44% + Menthol | 44.00% | 5.000 | |
| Huperzine A | 97.00% | 0.100 | |

| | | | RM supplied |
|---|---|---|---|
| | | Dosage | by (Capsugel |
| Excipients | Concentration | (mg) | or Customer) |
| Medium Chain Triglycerides | 100.00% | | Capsugel |
| Mixed Tocopherols | 90.00% | | Capsugel |
| Glycerol Monostearate | 100.00% | | Capsugel |
| | Total weight: | 590.000 | mgs |

*Raw Material

| | | | RM* supplied |
|---|---|---|---|
| | Concen- | Dosage | by (Capsugel |
| | tration | (mg) | or Customer) |
| Actives | | | |
| Rhodiola Rosea 3% Rosavins | 3.00% | 100.000 | Capsugel |
| Caffeine Anhydrous 99% | 99.00% | 100.000 | Capsugel |
| Geranium Oil | 25.00% | 120.000 | Capsugel |
| Bacopa monnieri (leaf) | | 80.000 | Capsugel |
| White Willow Bark Extract | 15.00% | 50.000 | Capsugel |
| 15% Salicin | | | |
| Vincamine | | 10.000 | Capsugel |
| Vanillin | | 10.000 | |
| Nicotinamide Adenine | | 5.000 | |
| Dinucleotide Hydrate | | | |
| Peppermint Oil 44% + Menthol | 44.00% | 5.000 | |
| Huperzine A | 97.00% | 0.100 | |
| Excipients | | | |
| Medium Chain Triglycerides | 100.00% | | Capsugel |
| Mixed Tocopherols | 90.00% | | Capsugel |
| Glycerol Monostearate | 100.00% | | Capsugel |
| | Total weight: | 590.000 | mgs |

*Raw Material

| | | | RM* supplied |
|---|---|---|---|
| | Concen- | Dosage | by (Capsugel |
| Actives | tration | (mg) | or Customer) |
| Rhodiola Rosea 3% Rosavins | 3.00% | 100.000 | Capsugel |
| Caffeine Anhydrous 99% | 99.00% | 100.000 | Capsugel |
| Geranium Oil | 25.00% | 100.000 | Capsugel |
| Bacopa monnieri | | 100.000 | Capsugel |
| White Willow Bark Extract 15% | 15.00% | 50.000 | Capsugel |
| Salicin | | | |
| Vincamine | | 10.000 | Capsugel |
| Vanillin | | 10.000 | |
| Nicotinamide Adenine Dinucleotide | | 5.000 | |
| Hydrate | | | |
| Peppermint Oil 44% + Menthol | 44.00% | 5.000 | |
| Huperzine A | 97.00% | 0.100 | |

| | | | RM supplied |
|---|---|---|---|
| | | Dosage | by (Capsugel |
| Excipients | Concentration | (mg) | or Customer) |
| Medium Chain Triglycerides | 100.00% | | Capsugel |
| Mixed Tocopherols | 90.00% | | Capsugel |
| Glycerol Monostearate | 100.00% | | Capsugel |
| | Total weight: | 590.000 | mgs |

*Raw Material

In preferred embodiments of the invention, the formulation may be administered orally in any suitable form, including, e.g., whole plants, powdered or pulverized plant material, extract, pill, capsule, granule, tablet or a suspension. Other forms of administration may also be used as considered to be appropriate be a person skilled in the art.

Any pharmaceutically acceptable carrier may be incorporated into the final composition. By the phrase, “pharmaceutically acceptable carrier,” it is meant any pharmaceutical carrier, such as the standard carriers described, e.g., Remington's Pharmaceutical Science, Eighteenth Edition, Mack Publishing company, 1990. Examples of suitable carriers are well known in the art and can include, but are not limited to, any of the standard pharmaceutical carriers such as a phosphate buffered saline solutions, phosphate buffered saline containing Polysorb 80, water, emulsions such as oil/water emulsion and various type of wetting agents. Other carriers may also include sterile solutions, tablets, coated tablets pharmaceutical and capsules. Typically such carriers contain excipients such as starch, milk, sugar, certain types of clay, gelatin, stearic acid or salts thereof, magnesium or calcium stearate, talc, vegetable fats or oils, gums, glycols. Such carriers can also include flavor and color additives or other ingredients. Compositions comprising such carriers are formulated by well known conventional methods. Generally excipients formulated with Rhodiola rosea are suitable for oral administration and do not deleteriously react with it, or other active components.

Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

A specific form of the formulation disclosed herein comprises the following ingredients in the amounts set out:

Rhodiola rosea: 100 mg

### Geranium Oil: 100 mg

### Anhydrous Caffeine: 100 mg

### Phosphatidylcholine: 140 mg

Salix alba: 50 mg Vinpocetine: 3 mg

Without further elaboration, it is believed that one skilled in the art can, using the preceding description, utilize the present invention to its fullest extent. The preceding preferred specific embodiments are, therefore, to be construed as merely illustrative, and not limiting the remainder of the disclosure in any way whatsoever.

The entire disclosures of all applications, patents and publications, cited above and in the figures are hereby incorporated by reference in their entireties. It should be noted that the numbers listed in the above portion of the description refer to the Natural Medicines Comprehensive Database found at the URL: www.naturaldatabase.com, the providers of this database being located at 3120 W. March Lane, PO Box 8190, Stockton, Calif. 95208, Tel: (209) 472-2244 Fax: (209) 472-2249.

Those skilled in the art will recognize, or be able to ascertain using no more than routine experimentation, many equivalents to the specific embodiments of the invention specifically described herein. Such equivalents are intended to be encompassed in the scope of the following claims.

### REFERENCES

Rhodiola rosea:
1927 Spasov A A, Wikman G K, Mandrikov V B, et al. A double-blind, placebo-controlled pilot study of the stimulating and adaptogenic effect of Rhodiola rosea SHR-5 extract on the fatigue of students caused by stress during an examination period with a repeated low-dose regimen. Phytomedicine 2000; 7:85-89.
3198 Petkov V D, Yonkov D, Mosharoff A, et al. Effects of alcohol aqueous extract from Rhodiola rosea L. roots on learning and memory. Acta Physiol Pharmacol Bulg 1986; 12:3-16.
6877 Darbinyan V, Kteyan A, Panossian A, et al. Rhodiola rosea in stress induced fatigue—a double blind cross-over study of a standardized extract SHR-5 with a repeated low-dose regimen on the mental performance of healthy physicians during night duty. Phytomedicine 2000; 7:365-71.
13028 Kelly G S. Rhodiola rosea: a possible plant adaptogen. Altern Med Rev 2001; 6:293-302.
13059 Akgul Y, Ferreira D, Abourashed E A, Khan I A. Lotaustralin from Rhodiola rosea roots. Fitoterapia 2004; 75:612-4.
15713 Wiedenfeld H, Dumaa M, Malinowski M, et al. Phytochemical and analytical studies of extracts from Rhodiola rosea and Rhodiola quadrifida. Pharmazie 2007; 62:308-11.
15714 Zhang L, Yu H, Sun Y, et al. Protective effects of salidroside on hydrogen peroxide-induced apoptosis in SH-SY5Y human neuroblastoma cells. Eur J. Pharmacol. 2007; 564:18-25.
15716 Perfumi M, Mattioli L. Adaptogenic and central nervous system effects of single doses of 3% rosavin and 1% salidroside Rhodiola rosea L. extract in mice. Phytother Res 2007; 21:37-43.
15718 Ma G, Li W, Dou D, et al. Rhodiolosides A-E, monoterpene glycosides from Rhodiola rosea. Chem Pharm Bull 2006; 54:1229-33.
16410 Bystritsky A, Kerwin L, Feusner J D. A pilot study of Rhodiola rosea (Rhodax) for generalized anxiety disorder (GAD). J Altern Complement Med 2008; 14:175-80.
16411 Shevtsov V A, Zholus B I, Shervarly V I, et al. A randomized trial of two different doses of a SHR-5 Rhodiola rosea extract versus placebo and control of capacity for mental work. Phytomedicine 2003; 10:95-105.

Vinpocetine:
1784 Thal L J, Salmon D P, Lasker B, et al. The safety and lack of efficacy of vinpocetine in Alzheimer's disease. J Am Geriatr Soc 1989; 37:515-20.
1786 Szakall S, Boros I, Balkay L, et al. Cerebral effects of a single dose of intravenous vinpocetine in chronic stroke patients: a PET study. J Neuroimaging 1998; 8:197-204.
1787 Hindmarch I, Fuchs H H, Erzigkeit H. Efficacy and tolerance of vinpocetine in ambulant patients suffering from mild to moderate organic psychosyndromes. Int Clin Psychopharmacol 1991; 6:31-43.
1789 Balestreri R, Fontana L, Astengo F. A double-blind placebo controlled evaluation of the safety and efficacy of vinpocetine in the treatment of patients with chronic vascular senile cerebral dysfunction. J Am Geriatr Soc 1987; 35:425-30.
1793 Miyazaki M. The effect of a cerebral vasodilator, vinpocetine, on cerebral vascular resistance evaluated by the Doppler ultrasonic technique in patients with cerebrovascular diseases. Angiology 1995; 46:53-8.
1800 Nicholson C D. Pharmacology of nootropics and metabolically active compounds in relation to their use in dementia. Psychopharmacology (Berl) 1990; 101:147-59.
1801 Akopov S E, Gabrielian E S. Effects of aspirin, dipyridamole, nifedipine and cavinton which act on platelet aggregation induced by different aggregating agents alone and in combination. Eur J Clin Pharmacol 1992; 42:257-9.
1802 Lohmann A, Dingler E, Sommer W, et al. Bioavailability of vinpocetine and interference of the time of application with food intake. Arzneimittelforschung 1992; 42:914-7.
10728 Grant J E, Veldee M S, Buchwald D. Analysis of dietary intake and selected nutrient concentrations in patients with chronic fatigue syndrome. J Am Diet Assoc 1996; 96:383-6.
10827 Szatmari S Z, Whitehouse P J. Vinpocetine for cognitive impairment and dementia. Cochrane Database Syst Rev 2003; 1:CD003119.

Phosphatidylcholine:
5228 Ladd S L, Sommer S A, LaBerge S, Toscano W. Effect of phosphatidylcholine on explicit memory. Clin Neuropharmacol 1993; 16:540-9.

Caffeine:
1238 Ascherio A, Zhang S M, Hernan M A, et al. Prospective study of caffeine intake and risk of Parkinson's disease in men and women. Proceedings 125th Ann Mtg Am Neurological Assn. Boston, Mass.: 2000; October 15-18:42 (abstract 53).
2718 Migliardi J R, Armellino J J, Friedman M, et al. Caffeine as an analgesic adjuvant in tension headache. Clin Pharmacol Ther 1994; 56:576-86.
2720 Rees K, Allen D, Lader M. The influences of age and caffeine on psychomotor and cognitive function. Psychopharmacology (Berl) 1999; 145:181-8.
2722 Nurminen M L, Niittynen L, Korpela R, Vapaatalo H. Coffee, caffeine and blood pressure: a critical review. Eur J Clin Nutr 1999; 53:831-9.
6022 Ross G W, Abbott R D, Petrovitch H, et al. Association of coffee and caffeine intake with the risk of parkinson disease. JAMA 2000; 283:2674-9.
6370 Sinclair C J, Geiger J D. Caffeine use in sports. A pharmacological review. J Sports Med Phys Fitness 2000; 40:71-9.
8646 Bell D G, Jacobs I, Ellerington K. Effect of caffeine and ephedrine ingestion on anaerobic exercise performance. Med Sci Sports Exerc 2001; 33:1399-403.
10201 Chen J F, Xu K, Petzer J P, et al. Neuroprotection by caffeine and A(2A) adenosine receptor inactivation in a model of Parkinson's disease. J Neurosci 2001; 21:RC143.
10205 Kamimori G H, Penetar D M, Headley D B, et al. Effect of three caffeine doses on plasma catecholamines and alertness during prolonged wakefulness. Eur J Clin Pharmacol 2000; 56:537-44.
11743 Smith A. Effects of caffeine on human behavior. Food Chem Toxicol 2002; 40:1243-55.
11832 Institute of Medicine. Caffeine for the Sustainment of Mental Task Performance: Formulations for Military Operations. Washington, D.C.: National Academy Press, 2001. Available at: http://books.nap.edu/books/0309082587/html/index.html.
12374 Lane J D, Barkauskas C E, Surwit R S, Feinglos M N. Caffeine impairs glucose metabolism in type 2 diabetes. Diabetes Care 2004; 27:2047-8.
13733 Acheson K J, Gremaud G, Meirim I, et al. Metabolic effects of caffeine in humans: lipid oxidation or futile cycling? Am J Clin Nutr 2004; 79:40-6.

Salix alba:
6456 Chrubasik S, Eisenberg E, Balan E, et al. Treatment of low back pain exacerbations with willow bark extract: a randomized double-blind study. Am J Med 2000; 109:9-14.
12476 Fiebich B L, Chrubasik S. Effects of an ethanolic salix extract on the release of selected inflammatory mediators in vitro. Phytomedicine 2004; 11:135-8.
12804 Chrubasik S, Kunzel O, Model A, et al. Treatment of low back pain with a herbal or synthetic anti-rheumatic: a randomized controlled study. Willow bark extract for low back pain. Rheumatology (Oxford) 2001; 40:1388-93.
12810 Krivoy N, Pavlotzky E, Chrubasik S, et al. Effect of salicis cortex extract on human platelet aggregation. Planta Med 2001; 67:209-12.

Huperzine-A:
3129 Wang X D, Zhang J M, Yang H H, Hu G Y. Modulation of NMDA receptor by huperzine A in rat cerebral cortex. Chung Kuo Yao Li Hsuch Pao 1999; 20:31-5.
3131 Skolnick A A. Old Chinese herbal medicine used for fever yields possible new Alzheimer Disease therapy. JAMA 1997; 277:776.
3132 Wang H, Tang X C. Anticholinesterase effects of huperzine A, E2020, and tacrine in rats. Chung Kuo Yao Li Hsuch Pao 1998; 19:27-30.
3133 Xiong Z Q, Cheng D H, Tang X C. Effects of huperzine A on nucleus basalis magnocellularis lesion-induced spatial working memory deficit. Chung Kuo Yao Li Hsuch Pao 1998; 19:128-32.
3134 Ye J W, Cai J X, Wang L M, Tang X C. Improving effects of huperzine A on spatial working memory in aged monkeys and young adult monkeys with experimental cognitive impairment. J Pharmacol Exp Ther 1999; 288:814-9
3135 Wang T, Tang X C. Reversal of scopolamine-induced deficits in radial maze performance by (−)-huperzine A: comparison with E2020 and tacrine. Eur J Pharmacol 1998; 349:137-42.
3136 Cheng D H, Tang X C. Comparative studies of huperzine A, E2020, and tacrine on behavior and cholinesterase activities. Pharmacol Biochem Behav 1998; 60:377-86.
3137 Lallement G, Veyret J, Masqueliez C, et al. Efficacy of huperzine in preventing soman-induced seizures, neuropathological changes and lethality. Fundam Clin Pharmacol 1997; 11:387-94.
3141 Tang X C, De Sarno P, Sugaya K, Giacobini E. Effect of huperzine A, a new cholinesterase inhibitor, on the central cholinergic system of the rat. J Neurosci Res 1989; 24:276-85.
3561 Pepping J. Huperzine A. Am J Health Syst Pharm 2000; 57:530-4.

Brahmi (Bacopa monnieri):
10058 Stough C, Lloyd J, Clarke J, et al. The chronic effects of an extract of Bacopa monniera (Brahmi) on cognitive function in healthy human subjects. Psychopharmacology 2001; 156:481-4.
10059 Yadav S K, Jain A K, Tripathi S N, Gupta J P. Irritable bowel syndrome: therapeutic evaluation of indigenous drugs. Indian J Med Res 1989; 90:496-503.
10060 Samiulla D S, Prashanth D, Amit A. Mast cell stabilising activity of Bacopa monnieri. Fitoterapia 2001; 72:284-5.
10061 Kidd P M. A review of nutrients and botanicals in the integrative management of cognitive dysfunction. Altern Med Rev 1999; 4:144-61.

Cowhage (Mucuna pruriens):
15 McKevoy G K, ed. AHFS Drug Information. Bethesda, Md.: American Society of Health-System Pharmacists, 1998.
18 Gruenwald J, Brendler T, Jaenicke C. PDR for Herbal Medicines. 1st ed. Montvale, N.J.: Medical Economics Company, Inc., 1998.
6898 Anon. Epidemiological notes and reports: Mucuna pruriens-associated pruritus—New Jersey. MMWR Morb Mortal Wkly Rep 1985; 34:732-3.
7020 HP-200 in Parkinson's Disease study group. An alternative medicine treatment for Parkinson's disease: Results of a multicenter clinical trial. J Alt Comp Med 1995; 1:249-55.
7021 Infante M E, Perez A M, Simao M R, et al. Outbreak of acute toxic psychosis attributed to Mucuna pruriens. Lancet 1990; 336:1129.

Periwinkle (Vinca minor):
2 Blumenthal M, ed. The Complete German Commission E Monographs: Therapeutic Guide to Herbal Medicines. Trans. S. Klein. Boston, Mass.: American Botanical Council, 1998.
19 Brinker F. Herb Contraindications and Drug Interactions. 2nd ed. Sandy, Oreg.: Eclectic Medical Publications, 1998.
513 Agri Res Svc: Dr. Duke's phytochemical and ethnobotanical databases. www.ars-grin.gov/duke (Accessed 3 Nov. 1999).
1799 The Natural Pharmacist. Vinpocetine. www.tnp.com/substance.asp?ID=573. (Accessed 16 Dec. 1999).

Nicotinamide Adenine Dinucleotide Hydrate (NADH):
3082 Budavari S, ed. The Merck Index. 12th ed. Whitehouse Station, N.J.: Merck & Co., Inc., 1996.
3083 Bushehri N, Jarrell S T, Lieberman S, et al. Oral reduced B-nicotinamide adenine dinucleotide (NADH) affects blood pressure, lipid peroxidation, and lipid profile in hypertensive rats (SHR). Geriatr Nephrol Urol 1998; 8:95-100.
3085 Swerdlow R H. Is NADH effective in the treatment of Parkinson's disease? Drugs Aging 1998; 13:263-8.
3086 Kuhn W, Muller T, Winkel R, et al. Parenteral application of NADH in Parkinson's disease: clinical improvement partially due to stimulation of endogenous levodopa biosynthesis. J Neural Transmiss (Budapest) 1996; 103:1187-93.
3088 Vrecko K, Birkmayer J G, Krainz J. Stimulation of dopamine biosynthesis in cultured PC 12 phaeochromocytoma cells by the coenzyme nicotinamide adenine dinucleotide (NADH). J Neural Transm Park Dis Dement Sect 1993; 5:147-56.
3091 Vrecko K, Storga D, Birkmayer J G, et al. NADH stimulates endogenous dopamine biosynthesis by enhancing the recycling of tetrahydrobiopterin in rat phaeochromocytoma cells. Biochim Biophys Acta 1997; 1361:59-65.
8260 Bushehri N, Jarrell S T, Lieberman S, et al. Oral reduced B-nicotinamide adenine dinucleotide (NADH) affects blood pressure, lipid peroxidation, and lipid profile in hypertensive rats (SHR). Geriatr Nephrol Urol 1998; 8:95-100.
8267 Forsyth L M, Preuss H G, MacDowell A L, et al. Therapeutic effects of oral NADH on the symptoms of patients with chronic fatigue syndrome. Ann Allergy Asthma Immunol 1999; 82:185-91.

Vanilla (Vanillin):
6 The Review of Natural Products by Facts and Comparisons. St. Louis, Mo.: Wolters Kluwer Co., 1999.
11 Leung A Y, Foster S. Encyclopedia of Common Natural Ingredients Used in Food, Drugs and Cosmetics. 2nd ed. New York, N.Y.: John Wiley & Sons, 1996.

Peppermint Oil (Menthol):
6742 Micklefield G H, Greying I, May B. Effects of peppermint oil and caraway oil on gastroduodenal motility. Phytother Res 2000; 14:20-3.
6744 Beesley A, Hardcastle J, Hardcastle P T, Taylor C J. Influence of peppermint oil on absorptive and secretory processes in rat small intestine. Gut 1996; 39:214-9.
11775 Sagduyu K. Peppermint oil for irritable bowel syndrome. Psychosomatics 2002; 43:508-9.
11776 Nolen H W 3rd, Friend D R. Menthol-beta-D-glucuronide: a potential prodrug for treatment of the irritable bowel syndrome. Pharm Res 1994; 11:1707-11.
11780 Somerville K W, Richmond C R, Bell G D. Delayed release peppermint oil capsules (Colpermin) for the spastic colon syndrome: a pharmacokinetic study. Br J Clin Pharmacol 1984; 18:638-40.
11781 Davies S J, Harding L M, Baranowski A P. A novel treatment of postherpetic neuralgia using peppermint oil. Clin J Pain 2002; 18:200-2.
12730 Inoue T, Sugimoto Y, Masuda H, Kamei C. Antiallergic effect of flavonoid glycosides obtained from Mentha piperita L. Biol Pharm Bull 2002; 25:256-9.
12731 Akdogan M, Ozguner M, Aydin G, Gokalp O. Investigation of biochemical and histopathological effects of Mentha piperita Labiatae and Mentha spicata Labiatae on liver tissue in rats. Hum Exp Toxicol 2004; 23:21-8.
12732 Akdogan M, Ozguner M, Kocak A, et al. Effects of peppermint teas on plasma testosterone, follicle-stimulating hormone, and luteinizing hormone levels and testicular tissue in rats. Urology 2004; 64:394-8.
12733 Inoue T, Sugimoto Y, Masuda H, Kamei C. Effects of peppermint (Mentha piperita L.) extracts on experimental allergic rhinitis in rats. Biol Pharm Bull 2001; 24:92-5.
13398 Storr M, Sibaev A, Weiser D, et al. Herbal extracts modulate the amplitude and frequency of slow waves in circular smooth muscle of mouse small intestine. Digestion 2004; 70:257-64.
13413 Nair B. Final report on the safety assessment of Mentha Piperita (Peppermint) Oil, Mentha Piperita (Peppermint) Leaf Extract, Mentha Piperita (Peppermint) Leaf, and Mentha Piperita (Peppermint) Leaf Water. Int J Toxicol 2001; 20:61-73.
13414 Iscan G, Kirimer N, Kurkcuoglu M, et al. Antimicrobial screening of Mentha piperita essential oils. J Agric Food Chem 2002; 50:3943-6.
13415 Pimentel M, Bonorris G G, Chow E J, Lin H C. Peppermint oil improves the manometric findings in diffuse esophageal spasm. J Clin Gastroenterol 2001; 33:27-31.
13447 Ramsewak R S, Nair M G, Stommel M, Selanders L. In vitro antagonistic activity of monoterpenes and their mixtures against ‘toe nail fungus’ pathogens. Phytother Res 2003; 17:376-9.

Geranium Oil:
4912 Electronic Code of Federal Regulations. Title 21. Part 182—Substances Generally Recognized As Safe.

1. A concentration and mental performance amplifying formulation comprising rhodiola rosea, geranium oil, vinpocetine, phosphatidylcholine, caffeine and Salix alba (White Willow Bark).

2. The concentration and mental performance amplifying formulation as claimed in claim 1 wherein there is present in each dosage: rhodiola rosea in an amount of from about 0.01 mg to about 600 mg; geranium oil in an amount of from about 0.01 mg to about 600 mg; vinpocetine in an amount of from about 0.01 mg to about 100 mg; phosphatidylcholine in an amount of from about 0.01 mg to about 600 mg; anhydrous caffeine in an amount of from about 0.01 mg to about 600 mg; and Salix alba (White Willow Bark) in an amount of from about 0.01 mg to about 600 mg.

3. The concentration and mental performance amplifying formulation as claimed in claim 1 wherein there is present in each dosage; 100 mg rhodiola rosea (3% Rosavins, 1% Salidrosides); 100 mg Geranium Oil; 3 mg vinpocetine; 140 mg phosphatidylcholine (Epicuron Lecithin 35% PPC); 100 mg anhydrous caffeine; and 50 mg Salix alba (white willow bark).

4. The concentration and mental performance amplifying formulation as claimed in claim 2 wherein the dosages comprise 1 to 2 dosages to a maximum of 6 dosages per day.

5. The concentration and mental performance amplifying formulation as claimed in claim 1 wherein there is present at least one excipient selected from glycerol monostearate, medium chain triglycerides and tocopherols.

6. A concentration and mental performance amplifying formulation comprising: rhodiola rosea, geranium oil, and at least one of anhydrous caffeine and nicotine.

7. The concentration and mental performance amplifying formulation as claimed in claim 6 further comprising: Vinpocetine, or Vincamine together with Huperzine-A, or Vinca minor (Periwinkle) together with Huperzine-A; PhosphatidylCholine or Bacopa monnieri (Brahmi) or Mucuna pruriens; Nicotinamide Adenine Dinucleotide Hydrate; and Salix alba (White Willow Bark).

8. The concentration and mental performance amplifying formulation as claimed in claim 7 further comprising: at least one flavouring agent selected from vanillin and peppermint oil.

9. The concentration and mental performance amplifying formulation as claimed in claim 8 wherein there is present at least one excipient selected from glycerol monostearate, medium chain triglycerides and tocopherols.

10. The concentration and mental performance amplifying formulation as claimed in claim 8 wherein there is present Rhodiola Rosea in an amount of about 0.01 mg to about 600 mg; Geranium Oil in an amount of about 0.01 mg to about 600 mg; nicotine in an amount of about 0.01 mg to about 30 mg; Vinpocetine in an amount of about 0.01 mg to about 100 mg; or Vincamine together with Huperzine-A in an amount of about 0.01 mg to about 100 mg; or Vinca minor (Periwinkle) together with Huperzine-A in an amount of about 0.01 mg to about 100 mg; PhosphatidylCholine or Bacopa monnieri (Brahmi) or Mucuna pruriens in an amount of about 0.01 mg to about 600 mg; Anhydrous Caffeine in an amount of about 0.01 mg to about 600 mg; Salix alba (White Willow Bark) in an amount of about 0.01 mg to about 600 mg; Nicotinamide Adenine Dinucleotide Hydrate: in an amount of about 0.01 mg to about 100 mg.; and Vanillin and/or Peppermint Oil: in an amount of about 0.01 mg to about 100 mg.

11. The concentration and mental performance amplifying formulation as claimed in claim 10 wherein the dosages comprise 1 to 2 dosages to a maximum of 6 dosages per day.

12. The concentration and mental performance amplifying formulation as claimed in claim 11 wherein the dosages are in the form of liquid containing capsules.

13. A method for amplifying concentration and mental performance in a human subject in need of such amplification, which comprises administering to said subject a formulation as claimed in claim 1.

14. A method for amplifying concentration and mental performance in a human subject in need of such amplification, which comprises administering to said subject a formulation as claimed in claim 6.

15. A method for amplifying concentration and mental performance in a human subject in need of such amplification, which comprises administering to said subject a formulation as claimed in claim 7.

16. A method for amplifying concentration and mental performance in a human subject in need of such amplification, which comprises administering to said subject a formulation as claimed in claim 8.

17. A method for amplifying concentration and mental performance in a human subject in need of such amplification, which comprises administering to said subject a formulation as claimed in claim 9.

18. A method for amplifying concentration and mental performance in a human subject in need of such amplification, which comprises administering to said subject a formulation as claimed in claim 10.

19. A method for amplifying concentration and mental performance in a human subject in need of such amplification, which comprises administering to said subject a formulation as claimed in claim 11.

20. A method for amplifying concentration and mental performance in a human subject in need of such amplification, which comprises administering to said subject a formulation as claimed in claim 12.
